SEQUENCE LISTING (55 sequences)

SEQ ID NO: 1 - protein, 198 aa, artificial sequence
  SARS-CoV-2 RBD
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 2 - protein, 127 aa, artificial sequence
  Re6A11
GSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTLVTVSSTS

SEQ ID NO: 3 - protein, 9 aa, artificial sequence
  Re6A11 CDR1
RTFSNDALG

SEQ ID NO: 4 - protein, 14 aa, artificial sequence
  Re6A11 CDR2
FVAAINWNSGTYYA

SEQ ID NO: 5 - protein, 18 aa, artificial sequence
  Re6A11 CDR3
AAASDYGLPREDFLYDYW

SEQ ID NO: 6 - protein, 131 aa, artificial sequence
  Re6B07
GSQVQLVESGGGLVQPGGSLRLSCAASGFTLDYYAIGWFRQAPGKEREGVSYIRSSDGTTYYADSVKGRFTISRDNAKNTVYLQMNSLKPEDTAVYYCAADEAYYSELGWESPWGWSYWGQGTRVTVSSTS

SEQ ID NO: 7 - protein, 9 aa, artificial sequence
  Re6B07 CDR1
FTLDYYAIG

SEQ ID NO: 8 - protein, 15 aa, artificial sequence
  Re6B07 CDR2
GVSYIRSSDGTTYYA

SEQ ID NO: 9 - protein, 21 aa, artificial sequence
  Re6B07 CDR3
AADEAYYSELGWESPWGWSYW

SEQ ID NO: 10 - protein, 131 aa, artificial sequence
  Re7E02
GSQVQLVESGGGLVQAGGSLRLSCAASGFTLDYYAIGWFRQAPGKEREGVSYIRSSDGTTYYADSVKGRFTISRDNAKNTVYLQMNSLKPEDTAVYYCAADEAYYSELGWESPWGWSYWGQGTQVTVSSTS

SEQ ID NO: 11 - protein, 9 aa, artificial sequence
  Re7E02 CDR1
FTLDYYAIG

SEQ ID NO: 12 - protein, 15 aa, artificial sequence
  Re7E02 CDR2
GVSYIRSSDGTTYYA

SEQ ID NO: 13 - protein, 21 aa, artificial sequence
  Re7E02 CDR3
AADEAYYSELGWESPWGWSYW

SEQ ID NO: 14 - protein, 131 aa, artificial sequence
  Re9C07
GSQVQLVESGGGLVQAGGSLRLSCAASGRTFSRYAMGWFRQAPGKEREFVAAITWNADTTYYADSVKGRFTISRDNAKNTVYLQMNSLKPEDTAVYYCAAGGNHYYSRSYYSSLEYDHWGQGTQVTVSSTS

SEQ ID NO: 15 - protein, 9 aa, artificial sequence
  Re9C07 CDR1
RTFSRYAMG

SEQ ID NO: 16 - protein, 15 aa, artificial sequence
  Re9C07 CDR2
FVAAITWNADTTYYA

SEQ ID NO: 17 - protein, 21 aa, artificial sequence
  Re9C07 CDR3
AAGGNHYYSRSYYSSLEYDHW

SEQ ID NO: 18 - protein, 129 aa, artificial sequence
  Re9D02
GSQVQLVESGGGLVQAGGSLRLSCAASGRTFSNYAMGWFRQAPGKEREFVAAISWGGDTTYYADSLKGRFTISRDNAKNTVYLQMNSLKPEDTAVYYCAADRGLSYYYDRVTEYDYWGQGTQVTVSSTS

SEQ ID NO: 19 - protein, 9 aa, artificial sequence
  Re9D02 CDR1
RTFSNYAMG

SEQ ID NO: 20 - protein, 15 aa, artificial sequence
  Re9D02 CDR2
FVAAISWGGDTTYYA

SEQ ID NO: 21 - protein, 19 aa, artificial sequence
  Re9D02 CDR3
AADRGLSYYYDRVTEYDYW

SEQ ID NO: 22 - protein, 127 aa, artificial sequence
  Re9F06
GSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTQVTVSSTS

SEQ ID NO: 23 - protein, 9 aa, artificial sequence
  Re9F06 CDR1
RTFSNDALG

SEQ ID NO: 24 - protein, 14 aa, artificial sequence
  Re9F06 CDR2
FVAAINWNSGTYYA

SEQ ID NO: 25 - protein, 18 aa, artificial sequence
  Re9F06 CDR3
AAASDYGLPREDFLYDYW

SEQ ID NO: 26 - protein, 129 aa, artificial sequence
  Re9G12
GSQVQLVESGGGLVQAGGSLRLSCAASGRTFSSYVMGWFRQAPGKEREFVAHISWSGDSTYYADSVKGRFTIFRDNAKNTAYLQMNSLKPEDTAVYYCAADRGASYYYTWASEYNYWGQGTQVTVSSTS

SEQ ID NO: 27 - protein, 9 aa, artificial sequence
  Re9G12 CDR1
RTFSSYVMG

SEQ ID NO: 28 - protein, 15 aa, artificial sequence
  Re9G12 CDR2
FVAHISWSGDSTYYA

SEQ ID NO: 29 - protein, 19 aa, artificial sequence
  Re9G12 CDR3
AADRGASYYYTWASEYNYW

SEQ ID NO: 30 - protein, 129 aa, artificial sequence
  Re11H04
GSQVQLVESGGGLVQPGGSLRLSCAASGFTLDYYTIAWFRQAPGKEREGVSCISGNDGSTYYADSVKGRFTISRDNAKNTVYLQMNSLKPEDTAVYYCAADRGESYYPFRPSEYHYWGQGTQVTVSSTS

SEQ ID NO: 31 - protein, 9 aa, artificial sequence
  Re11H04 CDR1
FTLDYYTIA

SEQ ID NO: 32 - protein, 15 aa, artificial sequence
  Re11H04 CDR2
GVSCISGNDGSTYYA

SEQ ID NO: 33 - protein, 19 aa, artificial sequence
  Re11H04 CDR3
AADRGESYYPFRPSEYHYW

SEQ ID NO: 34 - protein, 131 aa, artificial sequence
  Re5D06
GSQVQLVESGGGLVQPGGSLRLSCAASGITLDYYAIGWFRQAPGKEREGVSRIRSSDGSTNYADSVKGRFTMSRDNAKNTVYLQMNSLKPEDTAVYYCAYGPLTKYGSSWYWPYEYDYWGQGTQVTVSSTS

SEQ ID NO: 35 - protein, 9 aa, artificial sequence
  Re5D06 CDR1
ITLDYYAIG

SEQ ID NO: 36 - protein, 15 aa, artificial sequence
  Re5D06 CDR2
GVSRIRSSDGSTNYA

SEQ ID NO: 37 - protein, 21 aa, artificial sequence
  Re5D06 CDR3
AYGPLTKYGSSWYWPYEYDYW

SEQ ID NO: 38 - protein, 369 aa, artificial sequence
  Re6A11-spacer-RBD fusion
GSESEGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTLVTVSSTSEGSEGPESSDGSDSTDPGEQGEGADASDGSEGSSEGSEGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 39 - protein, 498 aa, artificial sequence
  His14-SUMO-Re6A11-spacer-RBD fusion
MSEEHHHHSGHHHTGHHHHSGSHHHGSEGSESSDSETNQEAEPESEPETEPETHINLKVSDGSSEIFFKIKKTTPLRRLMEAFAKRQGKEMDSLRFLYDGIRIQADQTPEDLDMEDNDIIEAQREQIGGGSESEGSQVQLVESGGGLVQAGGSLRLSCAASGRTFSNDALGWFRQAPRKEREFVAAINWNSGTYYADSVKGRFTISRDNAKNTVYLQMNSLKPEDTAVYSCAAASDYGLPREDFLYDYWGQGTLVTVSSTSEGSEGPESSDGSDSTDPGEQGEGADASDGSEGSSEGSEGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 40 - protein, 39 aa, artificial sequence
  Spacer
EGSEGPESSDGSDSTDPGEQGEGADASDGSEGSSEGSEG

SEQ ID NO: 41 - protein, 248 aa, artificial sequence
  DsbG protein
MLKKILLLALLPAIAFAEELPAPVKAIEKQGITIIKTFDAPGGMKGYLGKYQDMGVTIYLTPDGKHAISGYMYNEKGENLSNTLIEKEIYAPAGREMWQRMEQSHWLLDGKKDAPVIVYVFADPFCPYCKQFWQQARPWVDSGKVQLRTLLVGVIKPESPATAAAILASKDPAKTWQQYEASGGKLKLNVPANVSTEQMKVLSDNEKLMDDLGANVTPAIYYMSKENTLQQAVGLPDQKTLNIIMGNK

SEQ ID NO: 42 - protein, 130 aa, artificial sequence
  Re6H06
QVQLVESGGGLVQPGGSLRLSCAASGVTLDYYAIGWFRQAPGKEREGVSCTSSSDGSTYYADSVKGRFTISRDNAKNTVYLQMNSLKPEDTGVYYCAVVPQTYYGGKYYSQCTANGMDYWGKGTLVTVSS

SEQ ID NO: 43 - protein, 9 aa, artificial sequence
  Re6H06 CDR1
VTLDYYAIG

SEQ ID NO: 44 - protein, 15 aa, artificial sequence
  Re6H06 CDR2
GVSCTSSSDGSTYYA

SEQ ID NO: 45 - protein, 24 aa, artificial sequence
  Re6H06 CDR3
AVVPQTYYGGKYYSQCTANGMDYW

SEQ ID NO: 46 - skipped

SEQ ID NO: 47 - protein, 117 aa, artificial sequence
  Re21D01
EGSQVQLVESGGALVQPGGSLRLSCVASGFTFSSFAMGWYRQAPGKECEWVATITITGGSTNYADSVKGRFTISRDNAKNTVYLQMNSLKPEDTAVYYCNPDPGCRRGQGTQVTVSS

SEQ ID NO: 48 - protein, 9 aa, artificial sequence
  Re21D01 CDR1
FTFSSFAMG

SEQ ID NO: 49 - protein, 13 aa, artificial sequence
  Re21D01 CDR2
ATITITGGSTNYA

SEQ ID NO: 50 - protein, 8 aa, artificial sequence
  Re21D01 CDR3
NPDPGCRR

SEQ ID NO: 51 - protein, 119 aa, artificial sequence
  Re21H01
REGSQVQLVESGGALVQPGGSLRLSCVASGFTFSSFAMGWYRQAPGKECEWVATITITGGSTNYADSVKGRFTISRDNAKNTLYLQMNSLKPEDTAVYYCNPDPGCRGGGQGTQVTVSS

SEQ ID NO: 52 - protein, 9 aa, artificial sequence
  Re21H01 CDR1
FTFSSFAMG

SEQ ID NO: 53 - protein, 13 aa, artificial sequence
  Re21H01 CDR2
ATITITGGSTNYA

SEQ ID NO: 54 - protein, 9 aa, artificial sequence
  Re21H01 CDR3
NPDPGCRGG

SEQ ID NO: 55 - protein, 196 aa, artificial sequence
  SARS-CoV-2 S1 334-526 N343D || pDG3671
EGSNLCPFGEVFDATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCG

(19) United States
(12) Patent Application Publication
(10) Pub. No.: US 2024/0173398 A1
(43) Pub. Date: May 30, 2024

(54) FOLD PROMOTERS AND THEIR USE FOR THE PRODUCTION AND STABILIZATION OF POLYPEPTIDES

(71) Applicant: Max-Planck-Gesellschaft zur Förderung der Wissenschaften e.V., München (DE)

(72) Inventors: Dirk GÖRLICH, Göttingen (DE); Metin AKSU, Göttingen (DE); Thomas GÜTTLER, Göttingen (DE); Oleh RYMARENKO, Göttingen (DE); Renate REES, Gleichen (DE); Kathrin GREGOR, Dransfeld (DE); Waltraud TAXER, Northeim (DE)

(73) Assignee: Max-Planck-Gesellschaft zur Förderung der Wissenschaften e.V., München (DE)

(21) Appl. No.: 18/262,981

(22) PCT Filed: Jan. 28, 2022
(86) PCT No.: PCT/EP2022/052087

§ 371 Date: Jul. 26, 2023

(30) Foreign Application Priority Data
Jan. 28, 2021 (EP) 21154077.8

(51) Int. Cl. A61K 39/215; A61K 39/00; C07K 14/005; C07K 16/10; C12N 7/00

(52) U.S. Cl. CPC A61K 39/215; C07K 14/005; C07K 16/1003; C12N 7/00; A61K 2039/6031; C07K 2317/569; C12N 2770/20022; C12N 2770/20034; C12N 2770/20052

(57) ABSTRACT

The present invention relates to the recombinant production of a protein of interest in a prokaryotic host cell or eukaryotic host cell wherein the protein of interest is obtained in a correctly folded and stable form. The protein of interest may be a difficult-to-make polypeptide for use as a vaccine or a pharmaceutical. The protein of interest is co-expressed with or fused to a ‘fold promoter’, which may be a VHH antibody recognizing the said protein.

The present invention relates to the recombinant production of a protein of interest in a prokaryotic or eukaryotic host cell wherein the protein of interest is obtained in a correctly folded and stable form. The protein of interest may be a difficult-to-make polypeptide for use as a vaccine or a pharmaceutical. The protein of interest is co-expressed with or fused to a ‘fold promoter’, which may be a VHH antibody recognizing the said protein.

### BACKGROUND

Newly synthesized proteins typically need to fold to become functional. This folding process involves forming (hydrogen-bonded) secondary structures (such as α-helices and β-sheets) and the burial of hydrophobic residues into the protein's hydrophobic core to acquire their tertiary structure (for review see: (Balchin et al., 2020; Trombetta and Parodi, 2003). Many secretory proteins, such as immunoglobulins, serum albumin, or RNase A, also form disulfide bonds from initially reduced cysteines. A failure to fold correctly leads to non-functional proteins, aggregation, and/or rapid degradation by dedicated proteases. Many proteins assemble into multi-subunit complexes (quaternary structure), examples being immunoglobulins or hemoglobin. Subunits, which cannot find an appropriate partner subunit, are typically eliminated by cellular quality control systems.

Protein folding is typically catalyzed by dedicated folding enzymes called chaperones, whereby the available chaperone repertoire depends on where folding occurs. The eukaryotic cytosol harbors numerous chaperones, including members of the Hsp70 and Hsp90 ATPases and the TRIC complex, which confines folding intermediates into a large folding chamber and releases them when folding is complete. Folding can occur already during synthesis (co-translational folding) or in a posttranslational manner.

Secretory proteins are transported in an unfolded state through a so-called translocon into the lumen of the endoplasmic reticulum (ER). These include numerous proteins used in pharmaceutical applications, such as immunoglobulins, immune-regulatory factors, or hormones. The folding environment of the ER is rather different from the cytosolic one: while the cytosol is reducing, the ER lumen allows stabilizing disulfide bonds to form—catalyzed by a set of specific enzymes that includes protein disulfide isomerases (PDIs). Likewise, folding inside the ER is often coupled to another ER-specific posttranslational modification, namely N-glycosylation, which enables glycosylation-assisted folding mechanisms involving lectin-like molecules. The ER is also capable of ATP-dependent protein folding, using ER-specific Hsp70 and Hsp90-type family members. However, the ER lacks GroEL/Hsp60/TRIC complex-type chaperones typical of the cytosolic folding environment. After having passed quality control, secretory proteins exit the ER, traverse the Golgi, where additional modifications such as proteolytic processing or changes to the sugar chains might occur.

Protein folding in prokaryotes, such as Escherichia coli, shows similarities but also differences to eukaryotic folding. Instead of the TRIC complex, the E. coli cytosol contains a smaller and simpler GroE-GroL complex. Secretion in E. coli typically ends in the periplasmic space, which harbors disulfide-bonding enzymes, but lacks glycosylation, glycosylation-coupled folding, and ATP-dependent folding enzymes.

Recombinant expression of proteins is not only important for basic research, but also it is a key process in the area of biotechnology, in particular for the production of pharmaceutically relevant biologicals. Several expression systems are available for recombinant expression. Cytoplasmic expression in E.coli is the easiest to implement—because of the fast growth and straightforward genetic manipulation of this bacterium, because of economic growth media, and its safe handling. However, many target proteins do not fold correctly in the E.coli cytosol, in particular targets that usually fold in a secretory compartment and rely on correct disulfide-bonding and/or glycosylation-assisted folding.

There are several solutions to this problem, such as switching to another expression system, e.g., to secretory expression from mammalian CHO cells. However, the latter is far more time consuming and expensive, particularly when it comes to large-scale expressions. Another solution is to manipulate the cytosolic folding system of bacteria such as E.coli, for example, by making this folding environment less reducing and by ectopic cytoplasmic expression of a periplasmic disulfide isomerase (DsbC) (Bessette et al., 1999; Levy et al., 2001). Meanwhile, such strains are commercially available, e.g., the E.coli NEB Express Shuffle strain from New England Biolabs.

However, even when using bacterial strains that have a modified cytosolic folding system, certain polypeptides are still not correctly folded. Thus there is a need for further improving the folding of recombinant proteins produced in a bacterial host cell.

### SUMMARY OF THE INVENTION

A first aspect of the invention relates to a method for the recombinant production of a polypeptide of interest in a host cell, e.g. a prokaryotic host cell, or a eukaryotic host cell comprising the steps:
(a) providing a host cell comprising (i) a nucleic acid molecule encoding a fusion polypeptide comprising the polypeptide of interest and a fold promoter, or (ii) a first nucleic acid molecule encoding a polypeptide of interest and a second nucleic acid molecule encoding a fold promoter for the polypeptide of interest, wherein the fold promoter is a VHH antibody directed against the polypeptide of interest;
(b) cultivating the host cell under conditions where (i) the fusion polypeptide comprising the polypeptide of interest and the fold promoter is expressed or (ii) the polypeptide of interest and the fold promoter are co-expressed, and
(c) obtaining the polypeptide of interest in correctly folded form from the host cell.

In a particular embodiment, the polypeptide of interest is selected from a receptor binding domain (RBD) of a coronavirus spike protein, e.g. the RBD of the OC43, HKU1, 229E, NL63, SARS-CoV-1, MERS-COV or SARS-CoV-2 spike protein.

A further aspect of the invention relates to a nucleic molecule encoding a fusion polypeptide comprising a polypeptide of interest and a fold promoter for the polypeptide of interest, wherein the fold promoter is a VVH antibody directed against the polypeptide of interest.

A further aspect of the invention relates to a set of nucleic molecules comprising a first nucleic acid molecule encoding a polypeptide of interest and a second nucleic acid molecule encoding a fold promoter for the polypeptide of interest, wherein the fold promoter is a VHH antibody directed against the polypeptide of interest.

A further aspect of the invention relates to a host cell comprising a nucleic molecule or a set of nucleic acid molecules as described above.

In a particular embodiment, the host cell is a prokaryotic host cell, e.g. an E.coli cell.

A further aspect of the invention relates to a polypeptide selected from a receptor binding domain (RBD) of a coronavirus spike protein, e.g. the RBD of the OC43, HKU1, 229E, NL63, SARS-CoV-1, MERS-CoV or SARS-CoV-2 spike protein, in correctly folded form, particularly produced in a prokaryotic host cell.

In a particular embodiment, the polypeptide is the RBD of the SARS-CoV-2 spike protein in correctly folded form, particularly produced in a prokaryotic host cell.

A further aspect of the invention relates to a complex of an RBD of a coronavirus spike protein and a fold-enhancing VHH antibody.

A further aspect of the invention relates to an RBD as described above for use in medicine.

A further aspect of the invention relates to a polypeptide of interest for use as a vaccine, wherein the polypeptide of interest is attached to a heterologous amino acid sequence having a length of at least about 5 amino acids, which has a negative net charge of at least about −5.

### DETAILED DESCRIPTION

The present inventors explored the use of E.coli as a host cell for an economic recombinant production of the RBD (receptor-binding domain) of the SARS-CoV-2 spike protein. The RBD binds ACE2 during viral entry into target cells (Lan et al., 2020; Wang et al., 2020; Yan et al., 2020; Zhou et al., 2020a). Therefore, the RBD is a prime target for therapeutic intervention—be it for the production of neutralizing antibodies or as a constituent of vaccines. The RBD gets natively folded inside the ER lumen; it contains four structural disulfide bonds and gets N-glycosylated at two sites.

The inventors observed that the RBD failed to fold when expressed in the E. coli cytoplasm, even when using a strain that allows disulfide-bonding in the cytoplasm (NEB Shuffle). The small soluble fraction was then largely trapped in a non-productive complex with the chaperone GroE. This is in line with previous reports of failed folding of coronavirus RBDs in E. coli (Jegouic et al., 2020; Chen et al., 2005). Failure of correct folding was further confirmed by the finding that the soluble RBD fraction was not recognized by the super-neutralizing anti-RBD VHH antibody Re5D06, which is disclosed among a large set of other anti-RBD VHH antibodies in EP 20 188 541, EP 21 151 145, and EP 21 151 159, the contents of which are herein incorporated by reference.

Additional attempts of testing numerous variants of periplasmic RBD expression also gave unacceptably low yields. The still best variant was a fusion to the periplasmic disulfide interexchange protein G (DsbG), which yielded a soluble DsbG-RBD fusion that behaved in gel filtration like a folded protein. Nevertheless, the periplasmic expression workflow requires an osmotic shock protocol to release the periplasmic fraction, which cannot be easily implemented for an industrial-scale production; and yields were moderate at best.

The inventors discovered that a correct folding of the RBD domain could be enforced in the E.coli cytosol by its co-expression or fusion with “fold-promoting” anti-RBD VHH antibodies.

To this end, the inventors explored numerous ways to obtain a correctly folded RBD through expression in the E.coli cytosol. Eventually, the inventors tried the unconventional approach of testing systematically if co-expressing specific VHH antibodies could force the RBD to fold properly in the NEB Shuffle Express strain. Indeed, amongst 32 tested anti-RBD VHH antibodies, seven had the desired RBD-fold-promoting effect, namely: Re6A11, Re6B07, Re7E02, Re9C07, Re9D02, Re9F06, Re9G12 and Re11H04.

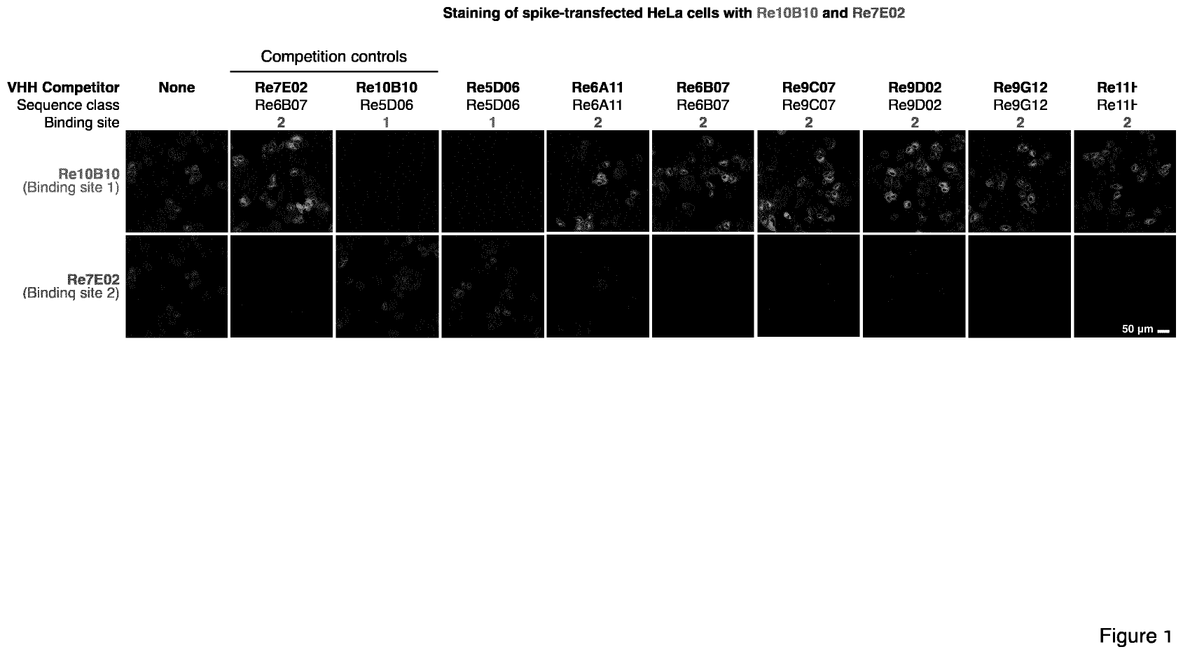

All of these seven VHH antibodies compete for the same RBD epitope (defined by Re7E02; see FIG. 1). By binding their epitope, it appears that these VHH antibodies stabilize an early folding intermediate and thereby block the divergence to non-productive folding paths. This “fold-promoter” epitope is non-overlapping with the epitope of the super-neutralizing VHH Re5D06 (FIG. 1), i.e., a fold-promoting VHH and Re5D06 can bind the same RBD molecule at the same time.

Accordingly, the present invention provides a covalent fusion polypeptide of a protein of interest, e.g. a coronavirus RBD and a fold promoting VHH antibody, a non-covalent complex of a protein of interest, e.g. a coronavirus RBD, and a fold promoting VHH antibody. The covalent fusion polypeptide optionally comprises further domains, e.g. a purification domain, e.g. an N-terminal cleavable purification tag, and/or a spacer located between the polypeptide of interest and the fold promoting VHH antibody.

The term “protein of interest” generally relates to any globular protein, protein fragment or protein domain that folds inefficiently when produced in a heterologous system.

The term “fold-enhancing VHH antibody” refers to a VHH antibody, which when co-expressed or fused to the protein of interest leads to an increase of the amount of correctly folded protein when expressed in a host cell, particularly in a prokaryotic host cell such as E. coli.

A “non-competing antibody” may be VHH antibody, or any other type of antibody or antigen-binding fragment thereof, and is directed against an epitope on the protein of interest, which is different from the epitope, against which the fold-enhancing VHH antibody is directed. Preferably, the non-competing antibody is a monovalent antibody, i.e. an antibody having a single antigen-binding site. More preferably, the non-competing antibody is a VHH antibody. The non-competing antibody and the fold-enhancing VHH antibody can bind to the protein of interest simultaneously. In certain embodiments, the non-competing antibody selectively binds to the protein of interest in correctly folded form, e.g. by binding to a conformational epitope on the protein of interest

A list of fold-enhancing VHH antibodies Re6A11, Re6B07, Re7E02, Re9C07, Re9D02, Re9F06, Re9G12, and Re11H04, and of the non-competing VHH antibody R6D05 and their CDR1, CDR2, and CDR3 sequences is shown in the following Table 1, as well as main epitope-binding Re6H06 and fold-enhancing VHH antibodies Re21D01 and Re21H01 as expressed in yeast:

TABLE 1

| | SEQ | | SEQ | | SEQ | | SEQ |
|---|---|---|---|---|---|---|---|
| | ID | | ID | | ID | | ID |
| VHH | NO. | CDR1 | NO. | CDR2 | NO. | CDR3 | NO. |
| Re6A11 | 2 | RTFSND | 3 | FVAAINWNSGTYY | 4 | AAASDYGLPREDFLYDYW | 5 |
| | | ALG | | A | | | |
| Re6B07 | 6 | FTLDYYA | 7 | GVSYIRSSDGTTY | 8 | AADEAYYSELGWESPWGW | 9 |
| | | IG | | YA | | SYW | |
| Re7E02 | 10 | FTLDYYA | 11 | GVSYIRSSDGTTY | 12 | AADEAYYSELGWESPWGW | 13 |
| | | IG | | YA | | SYW | |
| Re9C07 | 14 | RTFSRY | 15 | FVAAITWNADTTY | 16 | AAGGNHYYSRSYYSSLEYD | 17 |
| | | AMG | | YA | | HW | |
| Re9D02 | 18 | RTFSNY | 19 | FVAAISWGGDTTY | 20 | AADRGLSYYYDRVTEYDY | 21 |
| | | AMG | | YA | | W | |
| Re9F06 | 22 | RTFSND | 23 | FVAAINWNSGTYY | 24 | AAASDYGLPREDFLYDYW | 25 |
| | | ALG | | A | | | |
| Re9G12 | 26 | RTFSSY | 27 | FVAHISWSGDSTY | 28 | AADRGASYYYTWASEYNY | 29 |
| | | VMG | | YA | | W | |
| Re11H04 | 30 | FTLDYYT | 31 | GVSCISGNDGSTY | 32 | AADRGESYYPFRPSEYHY | 33 |
| | | IA | | YA | | W | |
| Re5D06 | 34 | ITLDYYAI | 35 | GVSRIRSSDGSTN | 36 | AYGPLTKYGSSWYWPYEY | 37 |
| | | G | | YA | | DYW | |
| Re6H06 | 42 | VTLDYY | 43 | GVSCTSSSDGST | 44 | AVVPQTYYGGKYYSQCTA | 45 |
| | | AIG | | YYA | | NGMDYW | |
| Re21D01 | 47 | FTFSSFA | 48 | ATITITGGSTNYA | 49 | NPDPGCRR | 50 |
| | | MG | | | | | |
| Re21H01 | 51 | FTFSSFA | 52 | ATITITGGSTNYA | 53 | NPDPGCRGG | 54 |
| | | MG | | | | | |

For cytoplasmic expression in E.coli, VHH are preferred that contain only a single disulfide bond, i.e. Re6A11, Re6B07, Re7E02, Re9C07, Re9D02, Re9F06, Re9G12, and Re11H04.

Re6A11, Re6B07, Re7E02, Re9C07, Re9D02, Re9F06, Re9G12, and Re11H04 as well as Re21D01 and Re21H01 are suitable for secretory expression in eukaryotes such as yeast.

The VHH antibody as described herein is particularly a monoclonal VHH antibody characterized by a specific amino acid sequence. The VHH antibody may be produced in a prokaryotic host cell, a yeast cell or a mammalian cell. In certain embodiments, the VHH antibody is non-glycosylated. In certain embodiments, the VHH antibody is glycosylated, wherein a carbohydrate structure may be derived from a glycosylation site introduced into the VHH sequence and/or from a fusion partner.

A VHH antibody is characterized by (i) a CDR3 sequence, (ii) a combination of a CDR1 sequence, a CDR2 sequence, and a CDR3 sequence, (iii) by a complete VHH sequence, or (iv) by competition with a specific reference antibody. Specific CDR and VHH sequences are provided in the Tables, Figures and the Sequence Listing.

According to the present invention, sequences related to the above sequences are encompassed. These related sequences are defined by having a minimum identity to a specifically indicated amino acid sequence, e.g. a CDR or VHH sequence. This identity is indicated over the whole length of the respective reference sequence and may be determined by using well known algorithms such as BLAST.

In particular embodiments, a related CDR3 sequence has an identity of at least 80% or at least 90% or at least 95% to a specifically indicated CDR3 sequence, e.g. a substitution of 1, 2, or 3 amino acids.

In particular embodiments, a related combination of a CDR1 sequence, a CDR2 sequence, and a CDR3 sequence has an identity of at least 80% or at least 90% or at least 95% to a specifically indicated combination of a CDR1 sequence, a CDR2 sequence, and a CDR3 sequence, e.g. a substitution of 1, 2, 3, 4, 5 or 6 amino acids by different amino acids.

In particular embodiments, a related VHH sequence has an identity of least 70%, at least 80%, at least 90%, at least 95% or at least 99% to a VHH sequence, e.g. a substitution of 1, 2, 3, 4, 5 or up to 20 amino acids.

Further, the invention refers to a VHH antibody, which competes with a specific VHH antibody disclosed herein for the binding to the SARS-CoV-2 spike protein S1 domain. In certain embodiments, a competing VHH antibody binds the same or an overlapping epitope on the SARS-CoV-2 spike protein S1 domain, particularly the receptor-binding domain (RBD) of the SARS-CoV-2 S1 domain as a specific VHH antibody disclosed herein. For example, the invention refers to a fold-enhancing VHH antibody, which competes with VHH antibodies Re6B07 or Re7E02.

Competition is measured either as an at least 90% loss of fluorescence staining of the polypeptide of interest, e.g. the SARS-CoV-2 spike protein by a fluorophore-labelled candidate VHH antibody in the presence of a 100-fold molar excess of an unlabelled competitor VHH antibody, e.g. Re7E02, or conversely as an at least 90% loss of polypeptide staining, e.g. spike protein-staining by a fluorophore-labelled VHH antibody, e.g. Re7E02 in the presence of a 100-fold molar excess of the unlabelled candidate VHH antibody as a competitor.

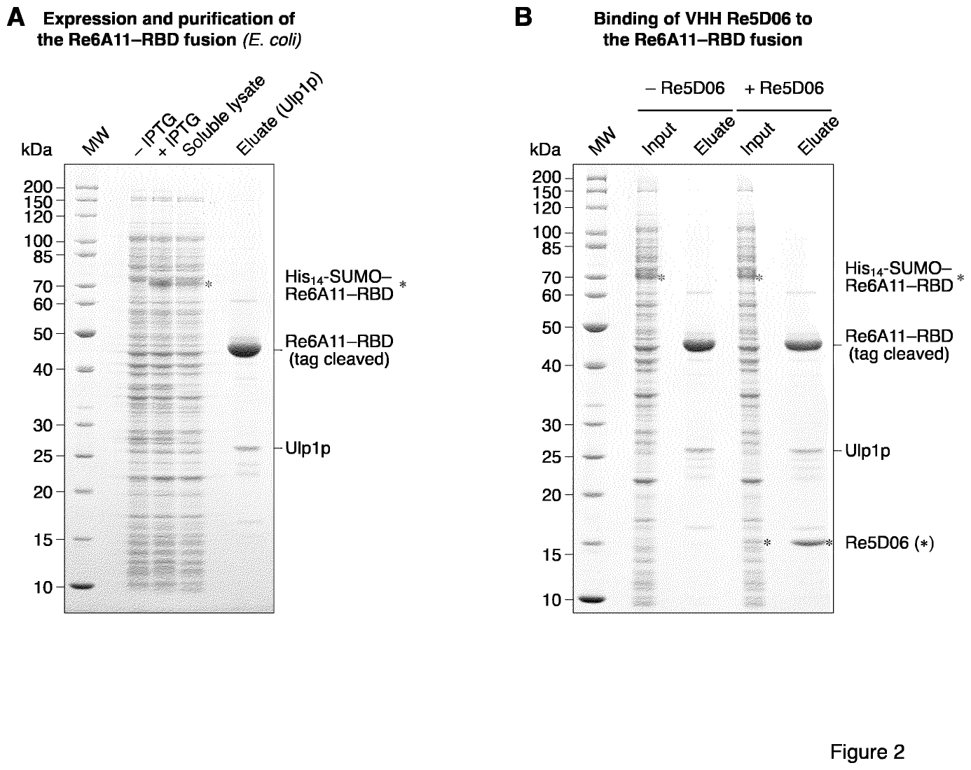

In their first experiments, the inventors co-expressed the VHH antibody and the RBD from two different plasmids. This was not quite ideal as the expression levels of the two polypeptides turned out to be different. Therefore, the presently preferred solution is to express the fold promoter and the RBD as a single fusion polypeptide comprising (i) optionally a cleavable N-terminal purification tag, e.g. the His14-SUMO module, (ii) the fold-promoting VHH, (iii) a spacer, e.g. a 39 residues long flexible, hydrophilic and negatively charged spacer, and (iv) the RBD. Such an entity (a His14-SUMO-VHH Re6A11-spacer-SARS-CoV-2-RBD fusion) was found to be well expressed in the cytoplasm of E. coli NEB Shuffle, soluble and purifiable in a single Ni (II) chelate step (FIG. 2A).

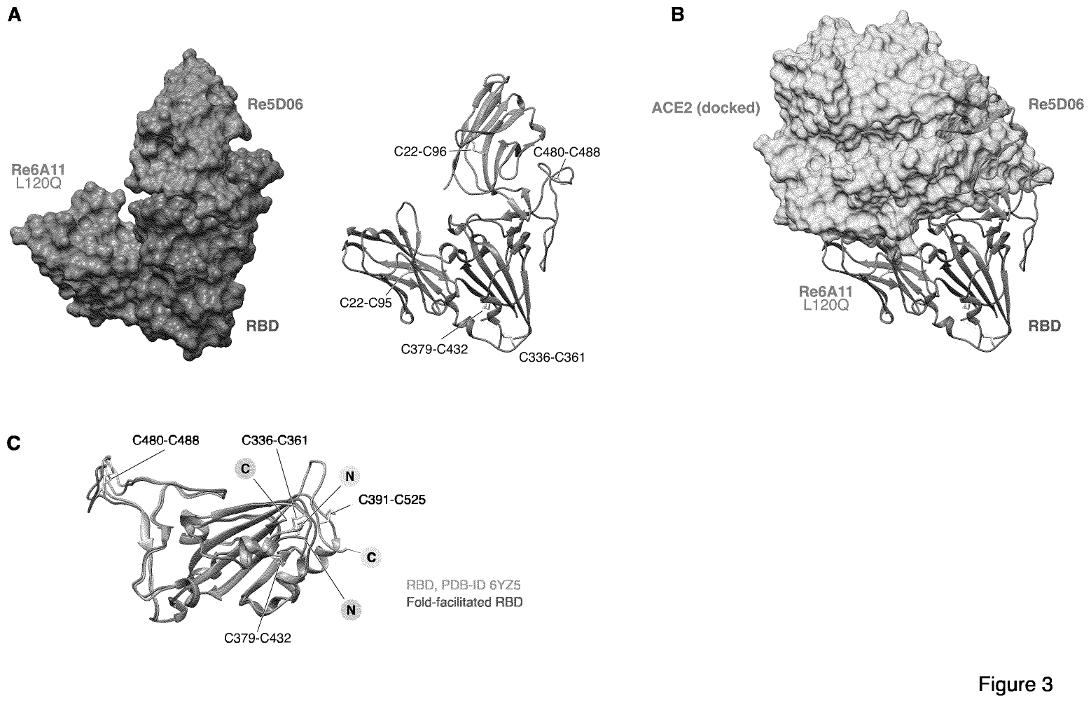

The interpretation of proper RBD-folding in the presence of those VHH antibodies rests on several lines of evidence. First, their co-expression or fusion increased soluble RBD expression. Second, it counteracted trapping by GroE. Third, binary fold-promoting VHH-RBD complexes or VHH-RBD fusions were easily obtained by co-expression and behaved, e.g., during gel filtration, like properly folded entities. Fourth, those binary complexes bound a non-competing antibody, e.g. complementary VHH Re5D06 with high affinity and specificity (FIG. 2B). As Re5D06 or an antibody competing therewith, e.g. as disclosed in EP 20 188 541, EP 21 151 145, and EP 21 151 159, recognizes a conformational epitope (see below FIG. 3), this again is evidence for proper folding.

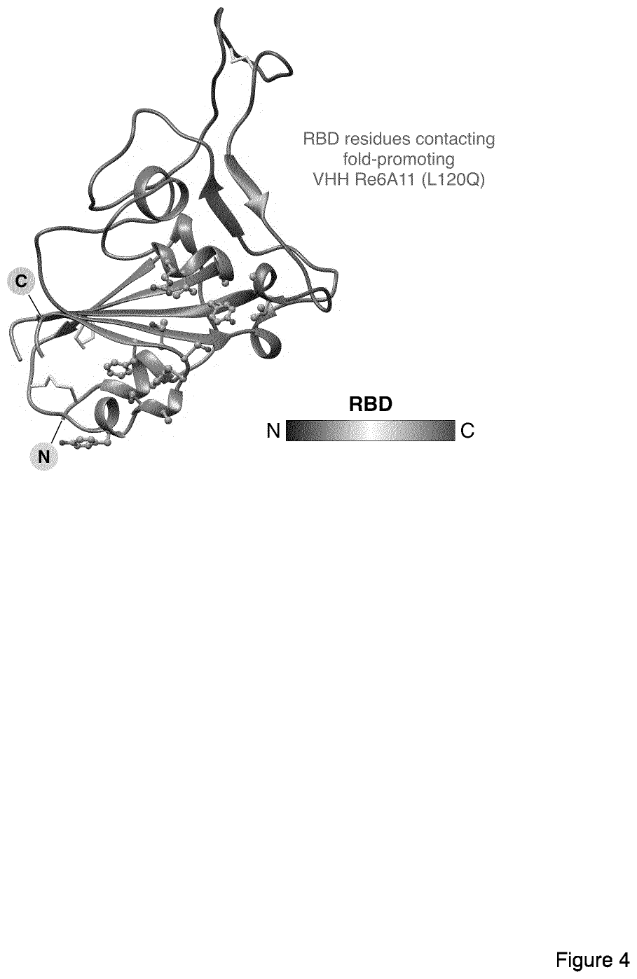

To obtain ultimate proof for correct folding of the RBD obtained under such expression conditions, the inventors crystallized a trimeric complex comprising the fold-promoting VHH antibody Re6A11, the RBD, and the neutralizing VHH antibody Re5D06 and solved the structure of this complex to a resolution of 1.75 Å (FIG. 3A-C). The structure indeed showed the correct fold of the RBD, including appropriate disulfide bonds. FIG. 3C visualizes this by overlaying the RBD from the fold-facilitator complex with a previously solved RBD structure (PDB 6YZ5). FIG. 4 shows the residues of the RBD, which are contacted by the fold-facilitator Re6A11. It appears as if the stabilization of this conformational epitope by VHH Re6A11 allows the RBD to complete the folding process properly. One can also rationalize the fold-promoting effect by considering that the VHH binding prevents the folding intermediate from getting trapped in a non-productive GroE complex. From yet a different perspective, one can see it as an artificial, stabilizing subunit of the RBD.

Another important detail is that the fold promoter produces only a comparably small clash with ACE2, which is sufficient to preclude ACE2-binding but leaves most of the ACE2-binding interface of the RBD exposed (FIG. 3B). In other words, the fold-promoter supports folding of the RBD without obstructing the interface that should be neutralized by vaccine-triggered antibodies.

The fold promoter Re6A11 not only leaves the best epitope for neutralization fully exposed but should even improve its presentation to the immune system because it prevents masking of this epitope by ACE2 (FIG. 3B). In fact, this might even reduce side effects of the immunization caused by the undesired binding of an RBD-vaccine to ACE2-presenting cells with subsequent antibody-binding/ opsonization of those cells.

In the aforementioned Re6A11-RBD fusion, the inventors linked the two entities with a long, negatively charged spacer of 39 residues in length and a net charge of -13. The length is needed to safely bridge the 56 Å distance between the C-terminus of Re6A11 and the N-terminus of the RBD. The negatively charged spacer serves, however, potentially yet another function, namely, to confer binding to cationic lipids or Al³⁺-based colloids that are typical components/carriers of vaccine formulations. This binding should concentrate the antigen on the surface of the particulate carriers and thus increase immunogenicity by presenting the antigen in a multivalent form.

The present invention provides a fusion polypeptide comprising a polypeptide of interest and a VHH antibody further comprising a heterologous amino acid sequence as a spacer having a length of at least about 5 amino acids and preferably up 100 amino acids, particularly about 5 to 50 amino acids. In certain embodiments, this polypeptide is selected from viral antigens that might trigger a neutralizing antibody response in humans or animals. The spacer has a negative net charge, particularly a negative net charge of at least about −5, more particularly a negative net charge of about −8 to about −30 or even more. The negative net charge of the spacer results from an excess of negatively charged amino acids therein. Preferably, the spacer has a content of at least about 15%, at least about 20% or least about 25% of negatively charged amino acids such as Asp and Glu. Further, the spacer may have a content of at least about 80%, or at least about 90% and up to about 100% of hydrophilic amino acids, particularly selected from Gly, Ser, Glu, Asp, Thr, Pro, and Gln.

The RBD co-expression with a fold-stabilizing VHH antibody as fold promoter or the RBD expression as a fusion with a fold-stabilizing VHH antibody a host cell like E. coli is thus a viable option to produce an anti-SARS-CoV-2 vaccine.

One can expand this concept to other vaccine candidates, such as RBDs from other coronaviruses that cause infections in humans (OC43, HKU1, 229E, NL63, SARS1, and MERS) or farm animals (e.g., swine and bovine coronaviruses).

The concept can be further expanded to other biotechnologically relevant biologicals, where a VHH can assist folding and production without interfering with the intended downstream application.

Further, the present invention provides a polypeptide of interest, particularly for use as a vaccine, wherein the polypeptide is attached to a heterologous amino acid sequence having a length of at least about 5 amino acids and preferably up 100 amino acids, particularly about 5 to 50 amino acids. In certain embodiments, this polypeptide is selected from viral antigens that might trigger a neutralizing antibody response in humans or animals. The heterologous amino acid sequence has a negative net charge, particularly a negative net charge of at least about −5, more particularly a negative net charge of about −8 to about −30 or even more. The negative net charge of the heterologous amino acid sequence results from an excess of negatively charged amino acids therein. Preferably, the heterologous amino acid sequence has a content of at least about 15%, at least about 20% or least about 25% of negatively charged amino acids such as Asp and Glu. Further, the heterologous amino acid sequence may have a content of at least about 80%, or at least about 90% and up to about 100% of hydrophilic amino acids, particularly selected from Gly, Ser, Glu, Asp, Thr, Pro, and Gln.

As indicated above, the heterologous spacer and the heterologous amino acid sequence have a negative net charge. This will increase the interaction with a positively charged compound, such as a polyamine, e.g. spermidine, a cationic polypeptide, e.g. protamine, a cationic lipid or an Al³⁺-based colloid or a particle comprising such a positively charged compound. Such a positively charged compound or particle is a typical component and/or carrier of a vaccine formulation. By means of this interaction, a non-covalent complex of the polypeptide and a positively charged compound or a particle comprising a positively charged compound will be generated.

Thereby, the polypeptide will be concentrated on the surface of a positively charged particulate carrier and thus increase immunogenicity by presenting the antigen in a multivalent form.

Thus, the invention provides a non-covalent complex of a polypeptide comprising a heterologous spacer or a heterologous amino acid sequence as described and positively charged compound including a particle comprising a positively charged compound. This complex is particularly useful as a component of immunogenic composition, e.g. an immunogenic composition for use as vaccine, e.g. in human medicine or in veterinary medicine, or for use in the production of antibodies, e.g. in an experimental animal.

### Fold-Promoting Effects of VHHs in Eukaryotic RBD Production in Yeast

To test if the fold-promoting effect of VHHs is restricted to production in E. coli or also applies to production in eukaryotes, the inventors expressed a SARS-CoV-2 RBD in the yeast Pichia pastoris as a secreted protein. This involves folding and disulfide-bonding in the endoplasmic reticulum as well as transport through the secretory pathway. This also involves quality control steps that need to be passed, for example, before the protein can exit the ER.

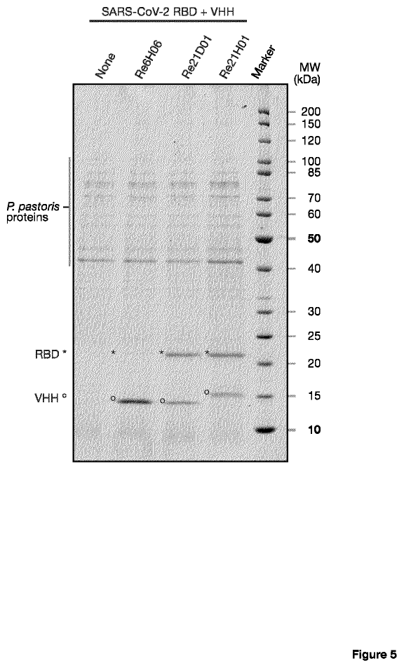

When expressed as a single protein, the RBD was not produced to detectable levels in the culture supernatant (FIG. 5). When the main epitope 1-binder (Re6H06) was co-expressed, only the VHH antibody but not the RBD became detectable. Co-expression of the epitope 2-binders Re21D01 or Re21H01, however, resulted in the appearance of a prominent RBD band along with VHH antibodies (FIG. 5). Subsequent biochemical and structural analysis revealed that the produced RBD had formed a tight complex either with Re21D01 or Re21H01. The subsequent crystallographic analysis confirmed correct disulfide bonding and proper folding of the RBD. Thus, co-expression of an appropriate VHH antibody with an otherwise inefficiently folding client can promote folding and passing the cellular quality control. This is effect is very specific as it requires the VHH to capture a specific epitope. The fold-promoting effect in Pichia is surprising since one should have assumed that the endoplasmic reticulum contains all factors and enzymes required for proper disulfide bonding. It can be explained by the yeast factors not being optimized for folding a component of a human pathogen. As with E. coli, it appears that stabilization of a folding intermediate prevents a divergence to a non-productive folding pathway. The effect can be exploited, for example, for the production of vaccines

In the following, particular embodiments of the specification are listed.

### EMBODIMENTS OF THE SPECIFICATION

1. A method for the recombinant production of a polypeptide of interest in a host cell, e.g. a prokaryotic host cell, or a eukaryotic host cell comprising the steps:
   (a) providing a host cell comprising (i) a nucleic acid molecule encoding a fusion polypeptide comprising the polypeptide of interest and a fold promoter, or (ii) a first nucleic acid molecule encoding a polypeptide of interest and a second nucleic acid molecule encoding a fold promoter for the polypeptide of interest, wherein the fold promoter is selected from:
      (i) a VHH antibody directed against the polypeptide of interest; and
      (ii) a periplasmic disulfide interexchange protein G (DsbG);
   (b) cultivating the host cell under conditions where (i) the fusion polypeptide comprising the polypeptide of interest and the fold promoter is expressed or (ii) the polypeptide of interest and the fold promoter are co-expressed, and
   (c) obtaining the polypeptide of interest in correctly folded form from the host cell.
2. The method of item 1, wherein the host cell is a prokaryotic host cell, e.g. a Gram-negative prokaryotic cell such as E. coli or a Gram-positive prokaryotic cell such as Bacillus ssp.
3. The method of item 1, wherein the host cell is a eukaryotic host cell, e.g. a yeast cell, an insect cell or a mammalian cell, in particular a yeast cell such as Pichia pastoris.
4. The method of any one of items 1-3, wherein polypeptide of interest is expressed as a fusion polypeptide.
5. The method of any one of items 1-4, wherein the nucleic acid molecule (i) encodes a fusion polypeptide further comprising an N-terminal purification tag, particularly a cleavable tag, e.g. a poly-His tag, a cleavable poly-His-SUMO tag or a cleavable poly-His-NEDD8 tag.
6. The method of any one of items 1-5, wherein the nucleic acid molecule (i) encodes a fusion polypeptide further comprising a heterologous spacer between the polypeptide of interest and the fold promoter.
7. The method of item 6, wherein the spacer has a length of at least about 5 amino acids and up to about 100 amino acids, particularly about 5 to about 50 amino acids.
8. The method of item 7, wherein the spacer has a negative net charge of at least -5, and particularly a content of at least about 15%, at least about 20% or least about 25% of negatively charged amino acids selected from Asp and Glu.
9. The method of any one of items 6-8, wherein the spacer has a content of at least about 80%, or at least about 90% and up to about 100% of hydrophilic amino acids, particularly selected from Gly, Ser, Glu, Asp, Thr, Pro, and Gln.
10. The method of any one of items 1-9, wherein the polypeptide of interest is expressed as a fusion polypeptide and wherein the polypeptide of interest is cleaved from the fold promoter or wherein the polypeptide is obtained as a fusion polypeptide.
11. The method of any one of items 1-3, wherein the polypeptide of interest and the fold promoter are expressed as separate polypeptides.
12. The method of any one of the preceding items, wherein the polypeptide of interest is the RBD of a coronavirus spike protein, e.g. the RBD of the SARS-Cov-1 spike protein, the RBD of the MERS-CoV spike protein, or the RBD of the SARS-CoV-2 spike protein.
13. The method of item 12, wherein the polypeptide of interest is the RBD of the SARS-CoV-2 spike protein, particularly having an amino acid sequence as shown in SEQ: ID NO. 1 or a sequence identity of at least 80%, at least 90% or at least 95% thereto.
14. The method of item 12 or 13, wherein the fold promoter is selected from a VHH antibody comprising
   (a) a CDR3 sequence as shown in SEQ ID NO. 5, 9, 13, 17, 21, 25, 29, 33, 50 or 54,
   (b) a CDR3 sequence, which has an identity of at least 80%, at least 90% or at least 95% to a CDR3 sequence of (a), or
   (c) a VHH antibody, which competes with a VHH antibody of (a) for the binding to the SARS-CoV-2 spike protein S1 domain, particularly the receptor-binding domain (RBD) of the SARS-CoV-2 S1 domain.
15. The method of any one of items 12-14, wherein the fold promoter is selected from a VHH antibody comprising
   (a) a combination of CDR1, CDR2 and CDR3 sequences as shown in SEQ ID NO. 3-5, 7-9, 11-13, 15-17, 19-21, 23-25, 27-29, 31-33, 47-50 or 51-54,
   (b) a combination of CDR1, CDR2 and CDR3 sequences which has an identity of at least 80%, at least 90% or at least 95% to a combination of CDR1, CDR2 and CDR3 sequences of (a), or
   (c) a VHH antibody, which competes with a VHH antibody of (a) for the binding to the SARS-CoV-2 spike protein S1 domain, particularly the receptor-binding domain (RBD) of the SARS-CoV-2 S1 domain.
16. The method of any one of items 12-15, wherein the fold promoter is selected from a VHH antibody comprising
   (a) a VHH sequence as shown in SEQ ID NO. 2, 6, 10, 14, 18, 22, 26, 30, 47 or 51,
   (b) a sequence which has an identity of at least 70%, at least 80%, at least 90%, at least 95% or at least 99% to a VHH sequence of (a), or
   (c) a VHH antibody, which competes with a VHH antibody of (a) for the binding to the SARS-CoV-2 spike protein S1 domain, particularly the receptor-binding domain (RBD) of the SARS-CoV-2 S1 domain. For expression in prokaryotic host cells VHH sequences as shown in SEQ ID NO. 2, 6, 10, 14, 18, 22, 26 or 30 are preferred; for expression in eukaryotic cells VHH sequences as shown in SEQ ID NO. 2, 6, 10, 14, 18, 22, 26, 30, 47 or 51, in particular 47 or 51 are preferred.
17. A nucleic molecule encoding a fusion polypeptide comprising a polypeptide of interest and a fold promoter for the polypeptide of interest, wherein the fold promoter is selected from:
   (i) a VVH antibody directed against the polypeptide of interest; and
   (ii) a periplasmic disulfide interexchange protein G (DsbG).
18. A vector comprising the nucleic acid molecule of item 17 in operative linkage with an expression control sequence.
19. A set of nucleic molecules comprising a first nucleic acid molecule encoding a polypeptide of interest and a second nucleic acid molecule encoding a fold promoter for the polypeptide of interest, wherein the fold promoter is selected from:
   (i) a VVH antibody directed against the polypeptide of interest; and
   (ii) a periplasmic disulfide interexchange protein G (DsbG).
20. A vector or a set of vectors comprising the first nucleic acid molecule and the second nucleic acid molecule of item 19 each in operative linkage with an expression control sequence.
21. A host cell comprising a nucleic acid molecule of item 17, a vector of item 18, a set of nucleic acid molecules of item 19 or a vector or a set of vectors of item 20.
22. The host cell of item 21, wherein the nucleic acid molecule encoding the polypeptide of interest is extrachromosomal.
23. The host cell of item 21, wherein the nucleic acid molecule encoding the polypeptide of interest is chromosomally integrated.
24. An RBD of a coronavirus spike protein, e.g. the RBD of the OC43, HKU1, 229E, NL63, SARS-CoV-1, MERS-COV or SARS-CoV-2 spike protein and variants thereof, in correctly folded form, particularly produced in a prokaryotic host cell.
25. An RBD of the SARS-CoV-2 spike protein, particularly having an amino acid sequence as shown in SEQ ID NO. 1 or 55 or a sequence identity of at least 80%, at least 90% or at least 95% thereto, in correctly folded form, particularly produced in a prokaryotic host cell.
26. The RBD of item 25, which binds to VHH antibody comprising
   (a) a CDR3 sequence as shown in SEQ ID NO. 37 or 45 or a CDR3 sequence, which has an identity of at least 80%, at least 90% or at least 95% thereto,
   (b) a combination of CDR1, CDR2 and CDR3 sequences as shown in SEQ ID NO. 35-37 or 43-45 or a combination of CDR1, CDR2 and CDR3 sequences, which has an identity of at least 80%, at least 90% or at least 95% thereto,
   (c) a VHH sequence as shown in SEQ ID NO. 34 or 42, or
   (d) a VHH antibody, which competes with a VHH antibody of (a), (b) or (c) for the binding to the SARS-CoV-2 spike protein S1 domain, particularly the receptor-binding domain (RBD) of the SARS-CoV-2 S1 domain.
27. A fusion protein of an RBD of a coronavirus spike protein, e.g. the RBD of the OC43, HKU1, 229E, NL63, SARS-CoV-1, MERS-COV or SARS-CoV-2 spike protein and variants thereof, and a fold-enhancing VHH antibody.
28. The fusion protein of item 27 comprising the RBD of the SARS-CoV-2 spike protein, particularly having an amino acid sequence as shown in SEQ ID NO. 1 or 57 or a sequence identity of at least 80%, at least 90% or at least 95% thereto, and a fold-enhancing VHH antibody.
29. A non-covalent complex of an RBD of a coronavirus spike protein, e.g. the RBD of the OC43, HKU1, 229E, NL63, SARS-COV-1, MERS-COV or SARS-CoV-2 spike protein, and a fold-enhancing VHH antibody.
30. The non-covalent complex of item 29 comprising the RBD of the SARS-CoV-2 spike protein, particularly having an amino acid sequence as shown in SEQ ID NO. 1 or 55 or a sequence identity of at least 80%, at least 90% or at least 95% thereto, and a fold-enhancing VHH antibody.
31. A fusion protein comprising an RBD of a coronavirus spike protein, e.g. the RBD of the OC43, HKU1, 229E, NL63, SARS-CoV-1, MERS-CoV or SARS-CoV-2 spike protein and variants thereof and a periplasmic disulfide interexchange protein G (DsbG), in correctly folded form, particularly produced in a prokaryotic host cell or eukaryotic host cell.
32. The fusion protein of item 31 comprising an RBD of the SARS-CoV-2 spike protein, particularly having an amino acid sequence as shown in SEQ ID NO. 1 or 55 or a sequence identity of at least 80%, at least 90% or at least 95% thereto, and a periplasmic disulfide interexchange protein G (DsbG), in correctly folded form, particularly produced in a prokaryotic host cell for SEQ ID NO. 1 and in a eukaryotic host cell for SEQ ID NO. 55.
31. An RBD of any one of items 24-26 or 29-30 for use in medicine.
32. An RBD of any one of items 24-26 or 29-30 for use as a vaccine.
33. An RBD of any one of items 24-26 or 29-30 for use in the production of antibodies, particularly neutralizing antibodies.
34. A fusion polypeptide comprising a polypeptide of interest and a VHH antibody further comprising a heterologous amino acid sequence as a spacer having a length of at least about 5 amino acids and preferably up 100 amino acids, which has a negative net charge and particularly a content of at least about 15%, at least about 20% or least about 25% of negatively charged amino acids selected from Asp and Glu.
35. A polypeptide for use as a vaccine, which is attached to a heterologous amino acid sequence having a length of at least about 5 amino acids and particularly up to about 100 amino acids, which has a negative net charge of at least -5 and particularly a content of at least about 15%, at least about 20% or least about 25% of negatively charged amino acids selected from Asp and Glu.
36. A non-covalent complex of a polypeptide of item 34 or 35 and a positively charged compound or a particle comprising a positively charged compound.

According to the invention VHH, CDR1, CDR2 and CDR3 sequences characterized by SEQ IDs NO. 42 to SEQ ID NO. 55 relate in particular to the expression in eukaryotic host cells, in particular yeast such as Pichia pastoris.

### EXAMPLES AND FIGURES

### FIG. 1: All Fold-Promoting VHHs Compete for the Same Epitope on the RBD

HeLa cells were transiently transfected to express the SARS-CoV-2 spike protein. Following fixation, cells were stained for 1 hour with fluorophore-labeled Re10B10 (5 nM, green) and Re7E02 (15 nM, red) in the presence of the indicated unlabeled VHH competitors. Competitor (150 nM) was added 20 min prior to the labeled nanobodies. Cells were imaged by confocal laser scanning microscopy. For each competitor, the binding site on the RBD (epitope 1 or 2) are indicated. Re10B10 and Re5D06 define the competition class for epitope 1, while the fold promoters Re6A11, Re6B07, Re7E02, Re9C07, Re9D02, Re9G12, and Re11H04 compete for epitope 2 and thus bind to a different site.

### FIG. 2: Soluble Expression and Purification of the Re6A11-RBD Fusion

A) An expression plasmid was constructed that encodes a fusion with an N-terminal His-SUMO tag, VHH-Re6A11, a 39 residues long acidic spacer (net charge: −13), and the SARS-CoV-2 RBD. E. coli NEB Express Shuffle was transformed with this plasmid, and plasmid-containing transformants were selected with 50 μg/ml kanamycin. Bacteria were grown in TB medium, and T5/lac-controlled expression was induced at 25° C. with 100 μM IPTG for 3 hours. Cells were recovered by centrifugation, resuspended in 50 mM Tris/HCl PH 7.5, 300 mM NaCl, 20 mM imidazole/HCl PH 7.5, and lysed by ultrasonication. The soluble fraction was obtained by ultracentrifugation. 45 ml soluble fraction (corresponding to 750 ml culture) was bound to a 1.2 ml Ni(II) EDTA-amide chelate column. Non-bound material was washed off with sonication buffer, and the Re6A11-RBD fusion was eluted by cleaving the His-SUMO-tag with the SUMO Ulp1 protease. Panel shows analysis of the experiment by SDS-PAGE (Coomassie-staining). Note the strong Re6A11-RBD fusion band in the eluted fraction. The yield was approximately 8 mg Re6A11-RBD fusion per liter of culture.

B) An analytical version of the experiment from panel a. Where indicated, 1 μM untagged Re5D06 was added to the binding reaction; elution was with lysis buffer containing 500 mM imidazole, which leaves the His14-SUMO fusion intact. Co-elution of Re5D06 with His14-SUMO-Re6A11-RBD indicates a specific RBD. Re5D06 interaction. The endogenous E. coli proteins present in the soluble starting lysate can be considered as negative controls for the binding reaction.

### FIG. 3: High-Resolution Crystal Structure of the Ternary Re9F06·RBD·Re5D06 Complex

The complex was produced by co-expressing all three components in the cytoplasm of E. coli NEB Shuffle Express, then purified by double-tag purification (Frey and Görlich, 2014a; Frey and Görlich, 2014b) followed by gel filtration. The homogeneous complex was crystallized, an x-ray diffraction dataset was recorded at the Swiss Light Source synchrotron, and the structure solved by molecular replacement to a resolution of 1.75 Å and an Rfree of 0.229.

A) Crystal structure of the Re9F06·RBD·Re5D06 complex in surface (left) or ribbon representation (right). The intramolecular disulfide bonds (shown as yellow sticks) are labeled.

B) Ribbon representation of the Re9F06·RBD·Re5D06 complex as in A, but with RBD-bound ACE2 shown as transparent brown surface. Docking of ACE2 is based on alignment of the RBD with the SARS-CoV-2 RBD·ACE2 complex (PDB ID 7KMS; (Zhou et al., 2020b); RMSD=0.986 Å). Note the frontal clash of ACE2 with Re5D06. The clash between Re9F06 and ACE2 is minor and caused by CDR2 and the VHH scaffold protruding into ACE2; despite the clash, Re9F06 and ACE2 contact different residues on the RBD.

C) Comparison of the RBD structure in the ternary Re9F06·RBD·Re5D06 complex with the structure of the RBD as reported by PDB 6YZ5. Note that the folds are identical, with only minor deviations in some flexible loop structures.

### FIG. 4: The Fold-Promoter Re6A11 Recognizes a Conformational RBD Epitope

The SARS-CoV-2 RBD from FIG. 3 is shown as a ribbon colored according to the indicated color gradient, with its N-terminus in blue and C-terminus in red. Disulfide bridges are depicted as yellow sticks. Side chains that interact with the fold-promoting VHH Re9F06 are shown in green, as ball-and-sticks.

### FIG. 5: Fold-Promoting VHH Antibodies Enhance the Secretory Production of a SARS-Cov-2 RBD in the Yeast Pichia pastoris

To test if the fold-promoting effect is restricted to production in E. coli or also applies to eukaryotes, the inventors expressed a SARS-CoV-2 RBD in the yeast Pichia pastoris. This domain comprised residues 334-526 of the S1 spike and carried an N343D mutation to eliminate an N-glycosylation site. The RBD was cloned under the control of a methanol-inducible AOX1 promoter. It was fused behind an Ost1 secretion signal (for transport into the ER) and an a-factor propeptide with a C-terminal Kex2 cleavage site. This expression cassette was genomically integrated using homologous recombination and selection for a G418 resistance marker.

Three additional RBD strains with co-integrated VHH expression cassettes (selected through Zeocin resistance marker) were constructed for co-expressing either the epitope 1-binder Re6H06 or the epitope 2-binders Re21D01 or Re21H01. The resulting strains were cultivated, induced for 48 hours with 0.5% methanol (added twice) before analyzing the pattern of secreted proteins in the culture supernatant by SDS-PAGE and Coomassie-staining.

The VHH antibodies expressed upon methanol induction as expected (marked by °. No secretion of RBD was evident without an additional VHH or when the main epitope-binder Re6H06 was co-expressed. However, a major RBD band (marked by *) was evident when Re21D01 or Re21H01 were co-expressed.

Sequences

SEQ ID NO. 1: SARS-CoV-2 RBD
PNITNLCPFGEVFNATRFASVYAWNRKRISNCVADYSVLYNSASFSTFKCYGVSPT
KLNDLCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFPLQSYGFQ
PTNGVGYQPYRVVVLSFELLHAPATVCGP
SEQ ID NO. 2: Re6A11
GSQVQLVESGGGLVQAGGSLRLSCAASGRTFSNDALGWFRQAPRKEREFVAAIN
WNSGTYYADSVKGRFTISRDNAKNTVYLQMNSLKPEDTAVYSCAAASDYGLPRED
FLYDYWGQGTLVTVSSTS
SEQ ID NO. 6: Re6B07
GSQVQLVESGGGLVQPGGSLRLSCAASGFTLDYYAIGWFRQAPGKEREGVSYIRS
SDGTTYYADSVKGRFTISRDNAKNTVYLQMNSLKPEDTAVYYCAADEAYYSELGW
ESPWGWSYWGQGTRVTVSSTS
SEQ ID NO. 10: Re7E02
GSQVQLVESGGGLVQAGGSLRLSCAASGFTLDYYAIGWFRQAPGKEREGVSYIRS
SDGTTYYADSVKGRFTISRDNAKNTVYLQMNSLKPEDTAVYYCAADEAYYSELGW
ESPWGWSYWGQGTQVTVSSTS
SEQ ID NO. 14: Re9C07
GSQVQLVESGGGLVQAGGSLRLSCAASGRTFSRYAMGWFRQAPGKEREFVAAIT
WNADTTYYADSVKGRFTISRDNAKNTVYLQMNSLKPEDTAVYYCAAGGNHYYSRS
YYSSLEYDHWGQGTQVTVSSTS
SEQ ID NO. 18: Re9D02
GSQVQLVESGGGLVQAGGSLRLSCAASGRTFSNYAMGWFRQAPGKEREFVAAIS
WGGDTTYYADSLKGRFTISRDNAKNTVYLQMNSLKPEDTAVYYCAADRGLSYYYD
RVTEYDYWGQGTQVTVSSTS
SEQ ID NO. 22: Re9F06
GSQVQLVESGGGLVQAGGSLRLSCAASGRTFSNDALGWFRQAPRKEREFVAAIN
WNSGTYYADSVKGRFTISRDNAKNTVYLQMNSLKPEDTAVYSCAAASDYGLPRED
FLYDYWGQGTQVTVSSTS
SEQ ID NO. 26: Re9G12
GSQVQLVESGGGLVQAGGSLRLSCAASGRTFSSYVMGWFRQAPGKEREFVAHIS
WSGDSTYYADSVKGRFTIFRDNAKNTAYLQMNSLKPEDTAVYYCAADRGASYYYT
WASEYNYWGQGTQVTVSSTS
SEQ ID NO. 30: Re11H04
GSQVQLVESGGGLVQPGGSLRLSCAASGFTLDYYTIAWFRQAPGKEREGVSCISG
NDGSTYYADSVKGRFTISRDNAKNTVYLQMNSLKPEDTAVYYCAADRGESYYPFR
PSEYHYWGQGTQVTVSSTS
SEQ ID NO. 34: Re5D06
GSQVQLVESGGGLVQPGGSLRLSCAASGITLDYYAIGWFRQAPGKEREGVSRIRS
SDGSTNYADSVKGRFTMSRDNAKNTVYLQMNSLKPEDTAVYYCAYGPLTKYGSS
WYWPYEYDYWGQGTQVTVSSTS
SEQ ID NO. 38: Re6A11-spacer-RBD fusion
GSESEGSQVQLVESGGGLVQAGGSLRLSCAASGRTFSNDALGWFRQAPRKEREF
VAAINWNSGTYYADSVKGRFTISRDNAKNTVYLQMNSLKPEDTAVYSCAAASDYGL
PREDFLYDYWGQGTLVTVSSTSEGSEGPESSDGSDSTDPGEQGEGADASDGSEG
SSEGSEGPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEGENCYFP
LQSYGFQPTNGVGYQPYRVVVLSFELLHAPATVCGP
SEQ ID NO. 39: His14-SUMO-Re6A11-spacer-RBD fusion
MSEEHHHHSGHHHTGHHHHSGSHHHGSEGSESSDSETNQEAEPESEPETEPETH
INLKVSDGSSEIFFKIKKTTPLRRLMEAFAKRQGKEMDSLRFLYDGIRIQADQTPEDL
DMEDNDIIEAQREQIGGGSESEGSQVQLVESGGGLVQAGGSLRLSCAASGRTFSN
DALGWFRQAPRKEREFVAAINWNSGTYYADSVKGRFTISRDNAKNTVYLQMNSLK
PEDTAVYSCAAASDYGLPREDFLYDYWGQGTLVTVSSTSEGSEGPESSDGSDSTD
PGEQGEGADASDGSEGSSEGSEGPNITNLCPFGEVFNATRFASVYAWNRKRISNC
VADYSVLYNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVCGP
SEQ ID NO. 40: Spacer
EGSEGPESSDGSDSTDPGEQGEGADASDGSEGSSEGSEG
SEQ ID NO. 41: DsbG
MLKKILLLALLPAIAFAEELPAPVKAIEKQGITIIKTFDAPGGMKGYLGKYQDMGVTIYL
TPDGKHAISGYMYNEKGENLSNTLIEKEIYAPAGREMWQRMEQSHWLLDGKKDAP
VIVYVFADPFCPYCKQFWQQARPWVDSGKVQLRTLLVGVIKPESPATAAAILASKD
PAKTWQQYEASGGKLKLNVPANVSTEQMKVLSDNEKLMDDLGANVTPAIYYMSKE
NTLQQAVGLPDQKTLNIIMGNK
SEQ ID NO. 55: SARS-CoV-2 S1 334-526 N343D||pDG3671
EGSNLCPFGEVFDATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDS
KVGGNYNYLYRLFRKSNLKPFERDISTEIYQAGSTPCNGVEGFNCYFPLQSYGFQP
TNGVGYQPYRVVVLSFELLHAPATVCG
SEQ ID NO. 42: Re6H06||pOR325
QVQLVESGGGLVQPGGSLRLSCAASGVTLDYYAIGWFRQAPGKEREGVSCTSSS
DGSTYYADSVKGRFTISRDNAKNTVYLQMNSLKPEDTGVYYCAVVPQTYYGGKYY
SQCTANGMDYWGKGTLVTVSS
SEQ ID NO. 47: Re21D01||pDG3678
EGSQVQLVESGGALVQPGGSLRLSCVASGFTFSSFAMGWYRQAPGKECEWVATI
TITGGSTNYADSVKGRFTISRDNAKNTVYLQMNSLKPEDTAVYYCNPDPGCRRGQ
GTQVTVSS
SEQ ID NO. 51: Re21H01||pDG3679
EGSQVQLVESGGALVQPGGSLRLSCVASGFTFSSFAMGWYRQAPGKECEWVATI
TITGGSTNYADSVKGRFTISRDNAKNTLYLQMNSLKPEDTAVYYCNPDPGCRGGG
QGTQVTVSS

### REFERENCES

Bessette, P. H., Åslund, F., Beckwith, J., and Georgiou, G. (1999). Efficient folding of proteins with multiple disulfide bonds in the Escherichia coli cytoplasm. Proceedings of the National Academy of Sciences 96, 13703-13708.
Chen, J., Miao, L., Li, J. M., Li, Y. Y., Zhu, Q. Y., Zhou, C. L., Fang, H. Q., and Chen, H. P. (2005). Receptor-binding domain of SARS-Cov spike protein: soluble expression in E. coli, purification and functional characterization. World J Gastroenterol 11, 6159-6164.
Frey, S., and Görlich, D. (2014a). A new set of highly efficient, tag-cleaving proteases for purifying recombinant proteins. J Chromatogr A 1337, 95-105.
Frey, S., and Görlich, D. (2014b). Purification of protein complexes of defined subunit stoichiometry using a set of orthogonal, tag-cleaving proteases. J Chromatogr A 1337, 106-115.
Jegouic, S. M., Loureiro, S., Thom, M., Paliwal, D., and Jones, I. M. (2020). Recombinant SARS-CoV-2 spike proteins for sero-surveillance and epitope mapping. bioRxiv
Lan, J., Ge, J., Yu, J., Shan, S., Zhou, H., Fan, S., Zhang, Q., Shi, X., Wang, Q., Zhang, L., and Wang, X. (2020). Structure of the SARS-CoV-2 spike receptor-binding domain bound to the ACE2 receptor. Nature 581, 215-220.
Levy, R., Weiss, R., Chen, G., Iverson, B. L., and Georgiou, G. (2001). Production of correctly folded Fab antibody fragment in the cytoplasm of Escherichia coli trxB gor mutants via the coexpression of molecular chaperones. Protein expression and purification 23, 338-347.
Wang, Q., Zhang, Y., Wu, L., Niu, S., Song, C., Zhang, Z., Lu, G., Qiao, C., Hu, Y., Yuen, K. Y., Wang, Q., Zhou, H., Yan, J., and Qi, J. (2020). Structural and Functional Basis of SARS-CoV-2 Entry by Using Human ACE2. Cell 181, 894-904.e9.
Yan, R., Zhang, Y., Li, Y., Xia, L., Guo, Y., and Zhou, Q. (2020). Structural basis for the recognition of SARS-CoV-2 by full-length human ACE2. Science 367, 1444-1448.
Zhou, P., Yang, X. L., Wang, X. G., Hu, B., Zhang, L., Zhang, W., Si, H. R., Zhu, Y., Li, B., Huang, C. L., Chen, H. D., Chen, J., Luo, Y., Guo, H., Jiang, R. D., Liu, M. Q., Chen, Y., Shen, X. R., Wang, X., Zheng, X. S., Zhao, K., Chen, Q. J., Deng, F., Liu, L. L., Yan, B., Zhan, F. X., Wang, Y. Y., Xiao, G. F., and Shi, Z. L. (2020a). A pneumonia outbreak associated with a new coronavirus of probable bat origin. Nature 579, 270-273.
Zhou, T., Tsybovsky, Y., Gorman, J., Rapp, M., Cerutti, G., Chuang, G. Y., Katsamba, P. S., Sampson, J. M., Schön, A., Bimela, J., Boyington, J. C., Nazzari, A., Olia, A. S., Shi, W., Sastry, M., Stephens, T., Stuckey, J., Teng, I. T., Wang, P., Wang, S., Zhang, B., Friesner, R. A., Ho, D. D., Mascola, J. R., Shapiro, L., and Kwong, P. D. (2020b). Cryo-EM Structures of SARS-CoV-2 Spike without and with ACE2 Reveal a pH-Dependent Switch to Mediate Endosomal Positioning of Receptor-Binding Domains. Cell Host Microbe 28, 867-879.e5.

1. A method for the recombinant production of a polypeptide of interest in a host cell, e.g. a prokaryotic host cell, or a eukaryotic host cell comprising the steps:
(a) providing a host cell comprising (i) a nucleic acid molecule encoding a fusion polypeptide comprising the polypeptide of interest and a fold promoter, or (ii) a first nucleic acid molecule encoding a polypeptide of interest and a second nucleic acid molecule encoding a fold promoter for the polypeptide of interest, wherein the fold promoter is a VHH antibody directed against the polypeptide of interest; and
(b) cultivating the host cell under conditions where (i) the fusion polypeptide comprising the polypeptide of interest and the fold promoter is expressed or (ii) the polypeptide of interest and the fold promoter are co-expressed, and
(c) obtaining the polypeptide of interest in correctly folded form from the host cell.

2. The method of claim 1, wherein the host cell is a prokaryotic host cell, e.g. a Gram-negative prokaryotic cell such as E. coli or a Gram-positive prokaryotic cell such as Bacillus ssp or a eukaryotic host cell such as yeast, in particular Pichia pastoris.

3. The method of claim 1, wherein the polypeptide of interest is expressed as a fusion polypeptide.

4. The method of claim 1, wherein the nucleic acid molecule (i) encodes a fusion polypeptide further comprising an N-terminal tag, particularly a cleavable tag, and/or a heterologous spacer between the polypeptide of interest and the fold promoter, wherein the spacer particularly has a negative net charge of at least −5, and more particularly a content of at least about 15%, at least about 20% or least about 25% of negatively charged amino acids selected from Asp and Glu.

5. The method of claim 1, wherein the polypeptide of interest and the fold promoter are expressed as separate polypeptides.

6. The method of claim 1, wherein the polypeptide of interest is the RBD of a coronavirus spike protein, e.g. the RBD of the SARS-Cov-1 spike protein and variants thereof, the RBD of the MERS-COV spike protein, or the RBD of the SARS-CoV-2 spike protein and variants thereof, and particularly the RBD of the SARS-CoV-2 spike protein, particularly having an amino acid sequence as shown in SEQ ID NO. 1 or SEQ ID NO. 55 or a sequence identity of at least 80%, at least 90% or at least 95% thereto.

7. The method of claim 6, wherein the fold promoter is selected from a VHH antibody comprising
(a) a CDR3 sequence as shown in SEQ ID NO. 5, 9, 13, 17, 21, 25, 29, 33, 50 or 54,
(b) a CDR3 sequence, which has an identity of at least 80%, at least 90% or at least 95% to a CDR3 sequence of (a), or
(c) a VHH antibody, which competes with a VHH antibody of (a) for the binding to the SARS-CoV-2 spike protein S1 domain, particularly the receptor-binding domain (RBD) of the SARS-CoV-2 S1 domain.

8. A nucleic molecule encoding a fusion polypeptide comprising a polypeptide of interest and a fold promoter for the polypeptide of interest, wherein the fold promoter is a VVH antibody directed against the polypeptide of interest.

9. A set of nucleic molecules comprising a first nucleic acid molecule encoding a polypeptide of interest and a second nucleic acid molecule encoding a fold promoter for the polypeptide of interest, wherein the fold promoter is a VVH antibody directed against the polypeptide of interest.

10. A host cell comprising a nucleic acid molecule of claim 8, or a set of nucleic acid molecules comprising a first nucleic acid molecule enclosing a polypeptide of interest and a second nucleic acid molecule encoding a fold promoter for the polypeptide of interest, wherein the fold promoter is a VVH antibody directed against the polypeptide of interest.

11. An RBD of the SARS-CoV-2 spike protein, particularly having an amino acid sequence as shown in SEQ ID NO. 1 or SEQ ID NO. 55 or a sequence identity of at least 80%, at least 90% or at least 95% thereto, in correctly folded form, particularly produced in a prokaryotic host cell, which binds to the non-competing VHH antibody Re5D06 having an amino acid sequence as shown in SEQ ID NO. 34 or 42.

12. A non-covalent complex comprising an RBD of a coronavirus spike protein, particularly the RBD of the SARS-CoV-2 spike protein, more particularly having an amino acid sequence as shown in SEQ ID NO. 1 or SEQ ID NO. 55 or a sequence identity of at least 80%, at least 90% or at least 95% thereto, and a fold-enhancing VHH antibody.

13. A covalent fusion protein comprising an RBD of a coronavirus spike protein, particularly the SARS-CoV-2 spike protein, more particularly having an amino acid sequence as shown in SEQ ID NO. 1 or SEQ ID NO. 55 or a sequence identity of at least 80%, at least 90% or at least 95% thereto, and a fold-enhancing VHH antibody

14. An RBD of claim 11, for use in medicine, particularly for use as a vaccine, or for use in the production of antibodies, particularly neutralizing antibodies.

15. A polypeptide for use as a vaccine, which is attached to a heterologous amino acid sequence having a length of at least about 5 amino acids and up to about 100 amino acids, and which has a negative net charge of at least −5 and particularly a content of at least about 15%, at least about 20% or least about 25% of negatively charged amino acids selected from Asp and Glu.
